(19) United States
(12) Patent Application Publication
(10) Pub. No.: US 2002/0081332 A1
(43) Pub. Date: Jun. 27, 2002

(54) Controlled release formulation of erythromycin or a derivative thereof

(76) Inventors: Ashok Rampal, Punjab (IN); Rajeev S. Raghuvanshi, New Delhi (IN); Manoj Kumar, Haryana (IN)

Correspondence Address: JAYADEEP R. DESHMUKH, ESQ., RANBAXY PHARMACEUTICALS INC., SUITE 2100, 600 COLLEGE ROAD EAST, PRINCETON, NJ, 08540, US

(21) Appl. No.: 09/941,970

(22) Filed: Aug. 29, 2001

(30) Foreign Application Priority Data
Aug. 29, 2000 (IN) 778/DEL/2000

(51) Int. Cl. A61K 9/62; A61K 9/20; A61K 31/715; A61K 31/7048

(52) U.S. Cl. 424/461; 514/29; 514/54

(57) ABSTRACT

The present invention relates to a controlled release pharmaceutical composition, suitable for once daily administration, of erythromycin or a derivative thereof and the process for its preparation. More preferably, it relates to a controlled release pharmaceutical composition of clarithromycin suitable for once daily administration.

### FIELD OF THE INVENTION

The present invention relates to a controlled release pharmaceutical composition, suitable for once daily administration, of erythromycin or a derivative thereof and the process for its preparation. More preferably, it relates to a controlled release pharmaceutical composition of clarithromycin suitable for once daily administration.

### BACKGROUND OF THE INVENTION

It is well known to those skilled in the art that the blood levels of drugs need to be maintained above a minimum effective level and below its minimum toxic level in order to obtain the desired therapeutic effects and to minimize side effects. Unfortunately, the pharmacokinetic properties (absorption, elimination and metabolism) of most drugs are such that they need to be administered three to four times a day. This kind of a dosing regimen is very inconvenient and leads to reduction in patient compliance. Reduction of dosing regimen from three times a day (tid) to twice daily (bid) to once a day results in increased convenience and comfort and therefore increased patient compliance. Controlled release formulations which are effective in maintaining the therapeutic blood levels over extended periods of time result in optimal therapy. They not only reduce the frequency of dosing, but they also reduce the severity and frequency of side effects, as they maintain substantially constant blood levels and avoid the fluctuations associated with the conventional immediate release formulations administered three to four times a day.

Erythromycin and its derivatives are known for their antibacterial activity against a number of organisms and are typically administered at least two to three times a day as immediate release compositions. In particular, the 6-O-methoxyerythromycin A (clarithromycin) which has been disclosed in U.S. Pat. No. 4,331,803 has to be administered at least twice daily for optimal effect.

Clarithromycin presents a peculiar problem for the formulator as it has greater solubility in the upper part of the gastrointestinal tract (GIT) but is very unstable at the acidic pH conditions in the GIT, and while its stability is good at alkaline pH of the large intestine (pH 6.0 to 8.0), its solubility is poor there. This results in poor bioavailability of clarithromycin.

U.S. Pat. No. 5,705,190 assigned to Abbott Laboratories describes controlled release compositions for such poorly soluble basic drugs comprising a water soluble alginate salt, a complex salt of alginic acid and an organic carboxylic acid to facilitate dissolution of the basic drug at a higher pH. As desired, the formulations described in the specification of this patent have an area under the plasma concentration—time curve (AUC) and minimum plasma concentration (Cmin) values which are substantially similar to those obtained by the immediate release tablets given twice daily. The maximum plasma concentration (Cmax) values, however, did not show the desired reduction and were similar to those for immediate release formulations.

Further, the total tablet weight of each tablet containing 500 mg drug as described in the examples of this invention is more than 900 mg, as substantial amounts of polymers are required for controlling the rate of drug release. A single tablet containing 1000 mg drug, when made according to this invention would weigh at least 1800 mg. This would be unacceptably large for human consumption, and two tablets of 500 mg strength each would be required for administrating the daily adult dose of 1000 mg clarithromycin.

U.S. Pat. No. 6,010,718 also assigned to Abbott describes an extended release pharmaceutical dosage form for clarithromycin, using from about 5 to about 50% by weight of a pharmaceutically acceptable polymer. The formulations described in this patent result not only in AUC and Cmin values similar to that of immediate release formulations administered twice daily, but also result in statistically significantly lower Cmax values. The total weight of the formulation as exemplified in this invention is close to 1000 mg for a tablet containing 500 mg drug. Once again, a single tablet would be unacceptably large at 2000 mg thus necessitating the administration of two tablets of 500 mg strength each for delivering the daily dose of 1000 mg clarithromycin.

U.S. Pat. No. 4,808,411 assigned to Abbott Laboratories claims a composition comprising from about 25% to about 95% of erythromycin A or a derivative thereof, and from about 5% to about 75% of a carbomer. The specification of this patent describes that the compositions made according to this invention provide palatable dosages of antibiotics. The pharmacokinetic properties, however, are not suitable for extended release and are substantially equivalent to commercially available immediate-release tablet and capsule formulations.

Accordingly, none of the oral controlled drug delivery systems heretofore described is completely satisfactory.

Our U.S. Pat. No. 6,261,601 describes a dosage form which is retained in the stomach for extended period of time and can deliver drugs to the stomach at a controlled rate. This delivery system is particularly useful for drugs which are stable in the acidic milieu of the stomach and which have a window of drug absorption from upper parts of gastro-intestinal in the stomach. The dosage form described in this application utilizes a unique combination of gel forming polymers, viscosity enhancing agents, hydrophilic polymers and gas generating agent. This application has however not explored the use of such systems for controlling the release of acid degradable drugs.

### SUMMARY OF THE INVENTION

It is an object of the present invention to provide a controlled release formulation for erythromycin or derivatives thereof that can deliver a daily dose of the drug in a single unit dosage form and wherein the rate controlling polymers are present at very small amounts of from about 0.1 to 4.0% w/w of the total weight of the dosage form, and wherein the delivery system maintains its physical integrity and a monolithic form when contacted with an aqueous media.

It is a further object of the present invention to provide a controlled release formulation for once daily administration of erythromycin or derivatives thereof, that contains a high dose medicament and is of an acceptable size which is convenient for oral administration. The use of small amounts of rate controlling polymers ensures that total weight of the dosage form is low and a single dosage unit is sufficient to provide the therapeutic dosage of the drug compared to two units which need to be administered if the teachings of the prior art are to be followed. The present invention provides obvious benefits with respect to better patient convenience and therefore patient compliance.

The present invention provides a controlled release formulation of erythromycin or derivatives thereof for once daily administration comprising an effective amount of the drug and about 0.1% w/w to about 4.0% w/w of one or more pharmaceutically acceptable rate controlling polymers.

More preferably, the present invention provides a controlled release formulation of clarithromycin for once daily administration comprising an effective amount of drug and from about 0.1% w/w to about 4.0% w/w of one or more pharmaceutically acceptable rate controlling polymers.

The present invention also provides a process for the preparation of a controlled release formulation of erythromycin or derivatives thereof for once daily administration comprising mixing a pharmaceutically effective amount of the drug with about 0.1% w/w to about 4.0% w/w of one or more pharmaceutically acceptable rate controlling polymers.

Clarithromycin used in accordance with the present invention comprises about 10% to about 90% w/w of the total formulation weight. More preferably, it constitutes about 50% to about 90% w/w of the formulation. The particle size of the drug may be reduced by techniques conventionally known in the art such as milling, pulverization, sieving, etc.

The pharmaceutically acceptable rate controlling polymers used in accordance with the present invention comprises of carbohydrate gums, polyuronic acid salts, cellulose ethers, acrylic acid polymers and mixtures thereof.

Carbohydrate gums may be selected from amongst xanthan gum, tragacanth gum, gum karaya, guar gum, acacia, gellan gum, locust bean gum, sclero gum and the like. These gums upon contact with the gastro intestinal fluid form a viscous gel and help in maintaining the tablet integrity and sustaining the release of the drug even when used in very small amounts. In preferred embodiments of this invention, the carbohydrate gum used is “xanthan gum” which is extraordinarily enzymatically resistant.

Examples of polyuronic acid salts that may be used in the present invention include alkali metal salts of alginic acid, alkali metal salts of pectic acid and mixtures thereof. In preferred embodiments of this invention, the water soluble salt of polyuronic acid is a salt of alginic acid, which is a mixture of two polyuronic acids, namely mannuoronic acid and gulucronic acid. Examples of alkali metal salts of alginic acid that may be used in the present invention include sodium alginate, potassium alginate, ammonium alginate, and the like. Importantly, when the pharmaceutical composition contains a water soluble salt of one or more polyuronic acids preferably a salt of alginic acid, it should be free of calcium ions.

The cellulose ethers used in accordance with the present invention include hydroxypropyl methylcellulose, hydroxypropyl cellulose, and the like. The polyacrylic acid polymers used may be such as is available under the brand name Carbopol (B.F. Goodrich, USA).

In addition to the rate controlling polymers, the composition may additionally contain about 6 to 50% w/w of other pharmaceutically acceptable excipients such as gas generating components, swelling agents, lubricants and fillers.

The gas generating components may constitute a single substance known to produce gas upon contact with gastric fluid, or may consist of a gas generating couple. Examples of the gas generating component that may be used in the present invention include carbonates, such as calcium carbonate or sodium glycine carbonate, bicarbonates, such as sodium hydrogen carbonate or potassium hydrogen carbonate, sulfites, such as sodium sulfite, sodium bisulfite or sodium metabisulfite, and the like. The gas generating component interacts with an acid source triggered by contact with water or simply gastric fluid to generate gas. These salts can be used alone or in combination with an acid source as a couple. Examples of organic acids that may be used as an acid source include citric acid or its salts such as sodium or calcium citrate, malic acid, tartaric acid, succinic acid, fumaric acid, maleic acid or their salts, ascorbic acid and its salts such as sodium or calcium ascorbate. The organic acid salts include mono or bialkali salts of organic acids having one or more than one carboxylic groups. Most preferably the gas generating agent is sodium bicarbonate. The gas generating components may be present at 5-45% w/w of the total weight of the formulation.

The swelling agent is one which is capable of swelling to greater than its original volume when coming into contact with an aqueous fluid such as the gastrointestinal fluid. Examples of such swelling agents that may be used in the present invention include cross-linked polyvinylpyrrolidone, cross-linked carboxymethylcellulose sodium, sodium starch glycolate, and the like. This class of compounds is also known as superdisintegrants and is present in an amount of from about 5 to about 25% w/w of the formulation. More preferably, it is present in an amount from about 10% to about 20% w/w of the total weight of the formulation.

The composition according to the present invention also contains pharmaceutically acceptable lubricants such as those selected from amongst talc, calcium stearate, magnesium stearate, polyethylene glycols, silicon dioxide, sodium lauryl sulfate, sodium stearyl fumarate and mixtures thereof.

The composition according to the present invention also contains fillers selected from amongst those conventionally used in the art such as lactose, starches, glucose, sucrose, mannitol, silicic acid and mixtures thereof. Fillers are present at about 5% to about 15% w/w of the formulation.

According to the present invention, the pharmaceutical composition can incorporate a high dose medicament. The amount of drug used in the composition varies from about 100 to 1000 mg and the total weight of the tablet does not exceed more than 1500 mg. The tablets made according to the present invention are unique as they carry a very high payload of the drug and use very small amounts of polymers for controlling the drug release while at the same time maintaining the integrity of the tablet for extended periods of time.

The composition according to the present invention may be formulated as a capsule or tablet. Most preferably, the composition is a tablet. The tablet formulation can be prepared by wet granulation, dry granulation, direct compression or by any other technique known in the pharmaceutical art. The tablet made according to the present invention may optionally be coated with a thin layer of a rapidly dissolving water soluble polymer or pharmaceutical excipient(s).

### DETAILED DESCRIPTION OF THE INVENTION

The examples given herein further illustrate the invention and are not intended to limit the scope of the invention.

### EXAMPLE I

TABLE 1.1

| Ingredients | mg/tab | Percent w/w of tablet weight |
|---|---|---|
| Clarithromycin equivalent to | 1000.0 | 81.0 |
| Sodium Alginate (LVCR) | 12.5 | 1.0 |
| Xanthan Gum | 37.5 | 3.0 |
| Cross-linked | 125.0 | 10.1 |
| polyvinylpyrrolidone (CL-PVP) | | |
| Magnesium Stearate | 12.5 | 1.0 |
| Talc | 20.0 | 1.6 |
| Sodium stearyl fumarate | 20.0 | 1.6 |
| Aerosil 200 | 8.0 | 0.70 |
| Purified water | Qs | qs |
| Total weight | 1235.5 | 100.0 |

Clarithromycin, sodium alginate, xanthan gum and CL-PVP were sieved through a British Standard Sieve (BSS) 44 mesh sieve and blended together followed by granulation with water. The granules were dried in a fluid bed drier at 60° C. for 20 minutes. The dried granules were sifted through a BSS 16 mesh sieve. The granules obtained were lubricated with the remaining ingredients namely talc, magnesium stearate, sodium stearyl fumarate and aerosil 200 and compressed to tablets.

The drug release from the tablets was monitored at pH 5.0 acetate buffer in USP apparatus I at 100 rpm and the results obtained are given in Table 1.2.

TABLE 1.2

| Time (h) | Cumulative Percent drug released |
|---|---|
| 1 | 19.0 |
| 2 | 27.0 |
| 4 | 40.0 |
| 6 | 45.0 |
| 8 | 51.0 |
| 10 | 55.0 |

The drug release was extended to more than 10 hours despite the use of only 4% w/w of the total rate controlling polymers indicating the efficacy of control. Release of only 55% of the drug in ten hours, was however unacceptably slow. The formulation was therefore modified to include a gas generating component to accelerate the rate of drug release as described in the next experiment.

### EXAMPLE 2

TABLE 2.1

| Ingredients | Mg/tab | Percent w/w of tablet weight |
|---|---|---|
| Clarithromycin equivalent to | 1000.0 | 75.0 |
| Sodium bicarbonate | 100.0 | 7.5 |
| Sodium Alginate (LVCR) | 50.0 | 3.7 |
| Cross-linked | 125.0 | 9.40 |
| polyvinylpyrrolidone (CL-PVP) | | |
| Magnesium Stearate | 12.5 | 0.90 |
| Talc | 20.0 | 1.5 |
| Sodium stearyl fumarate | 20.0 | 1.5 |
| Aerosil 200 | 8.0 | 0.6 |
| Purified Water | Qs | qs |
| Total weight | 1335.5 | 100.0 |

Tablets were made by the same process as described in Example 1 drug release (Table 2.2).

TABLE 2.2
Percent drug released in pH 5.0 acetate buffer
in USP apparatus I at 100 rpm.

| Time (h) | Cumulative Percent drug released |
|---|---|
| 1 | 21.0 |
| 2 | 30.0 |
| 4 | 48.0 |
| 6 | 78.0 |
| 8 | 85.0 |
| 10 | 87.0 |

### EXAMPLE 3

TABLE 3.1

| Ingredients | mg/tab | Percent w/w of tablet weight |
|---|---|---|
| Clarithromycin equivalent to | 1000.0 | 75.0 |
| Hydroxypropyl methylcellulose | 50.0 | 3.7 |
| (HPMC K100 MCR) | | |
| Sodium bicarbonate | 100.0 | 7.5 |
| Cross-linked | 125.0 | 9.4 |
| polyvinylpyrrolidone (CL-PVP) | | |
| Magnesium Stearate | 12.5 | 0.9 |
| Talc | 20.0 | 1.5 |
| Sodium Stearyl Fumarate | 20.0 | 1.5 |
| Aerosil 200 | 8.0 | 0.6 |
| Purified water | Qs | qs |
| Total weight | 1335.5 | 100.0 |

Tablets were made following the same process that described in Example 1, and subjected to dissolution testing in USP apparatus 1, at 100 rpm in pH 5.0 acetate buffer. The dissolution profile is given in Table 3.2.

TABLE 3.2

| Time (h) | Cumulative Percent drug released |
|---|---|
| 1 | 25.0 |
| 2 | 36.0 |
| 4 | 54.0 |
| 6 | 64.0 |
| 8 | 73.0 |
| 10 | 76.0 |

### EXAMPLE 4

TABLE 4.1

| Ingredients | mg/tab | Percent w/w of tablet weight |
|---|---|---|
| Clarithromycin equivalent to | 1000.0 | 84.3 |
| Hydroxypropyl methylcellulose | 12.5 | 1.1 |
| (HPMC K100MCR) | | |
| Sodium bicarbonate | 100.0 | 8.4 |
| Magnesium Stearate | 12.5 | 1.1 |
| Talc | 10.0 | 0.8 |
| Sodium Stearyl fumarate | 20.0 | 1.7 |
| Aerosil 200 | 5.0 | 0.4 |
| Purified water | qs | qs |
| Total weight | 1185.5 | 100.0 |

The tablets were made as described in Example 1. Only 1% HPMC was used as the rate controlling polymer. Tablets made according to the present example containing only 1% of rate controlling polymer were not only able to maintain their monolithic form, but were also capable of controlling the release of clarithromycin over an extended period of time as shown in Table 4.2.

TABLE 4.2

| Time (h) | Percent drug released |
|---|---|
| 1 | 7.0 |
| 2 | 12.0 |
| 4 | 16.0 |
| 6 | 24.0 |
| 8 | 53.0 |

### EXAMPLE 5

TABLE 5.1

| Ingredients | Mg/tab | Percent w/w of tablet weight |
|---|---|---|
| Clarithromycin equivalent to | 1019.0 | 80.3 |
| Sodium alginate (LVCR) | 12.5 | 1.1 |
| Xanthan Gum | 37.5 | 3.01 |
| Sodium bicarbonate | 100.0 | 8.0 |
| Magnesium Stearate | 20.0 | 1.6 |
| Talc | 20.0 | 1.6 |
| Sodium stearyl fumarate | 30.0 | 2.4 |
| Aerosil 200 | 5.0 | 0.4 |
| Purified water | qs | qs |
| Total weight | 1244.8 | 100.0 |

Tablets were made as described in Example 1 and Table 5.2 gives the dissolution profile of these tablets in pH 5.0 acetate buffer, USP apparatus I at 100 rpm.

TABLE 5.2

| Time (h) | Cumulative Percent drug released |
|---|---|
| 1 | 5 |
| 2 | 13 |
| 4 | 29 |
| 6 | 48 |
| 8 | 62 |
| 10 | 70 |

While the present invention has been described in terms of its specific embodiments, certain modifications and equivalents will be apparent to those skilled in the art and are intended to be included within the scope of the present invention.

1. A controlled release formulation of erythromycin A or a derivatives thereof, suitable for once daily administration, comprising a pharmaceutically effective amount of erythromycin and from about 0.1% to about 4% w/w of one or more pharmaceutically acceptable rate controlling polymers.

2. A controlled release formulation as described in claim 1, wherein the erythromycin A derivative is clarithromycin.

3. A controlled release formulation as described in claim 1 wherein erythromycin A or its derivative comprises about 10% w/w to about 90% w/w of the total tablet weight.

4. A controlled release formulation as described in claim 3 wherein erythromycin A or its derivative preferably comprises about 50% w/w to about 90 % w/w of the total tablet weight.

5. A controlled release formulation described in claim 1 wherein the pharmaceutically acceptable rate controlling polymer comprises of carbohydrate gum, polyuronic acid salt, cellulose ether, acrylic acid polymer, and mixtures thereof.

6. A controlled release formulation as described in claim 5 wherein the carbohydrate gum is selected from the group consisting of xanthan gum, tragacanth gum, gum karaya, guar gum, acacia, gellan gum, locust bean gum, sclero gum, and mixtures thereof.

7. A controlled release formulation as described in claim 5 wherein the polyuronic acid salt is selected from the group consisting of alkali metal salts of pectic acid, alkali metal salts of alginic acid, and mixtures thereof.

8. A controlled release formulation as described in claim 7 wherein the polyuronic acid salt is preferably sodium alginate.

9. A controlled release formulation as described in claim 5 wherein the cellulose ether are selected from the group consisting of hydroxypropyl methylcellulose, hydroxypropylcellulose, and mixtures thereof.

10. A controlled release formulation as described in claim 5 wherein the acrylic acid polymer is carbopol.

11. A monolithic controlled release formulation of clarithromycin comprising 100-1000 mg of clarithromycin, wherein the total weight of the dosage unit is not more than 1500 mg.

12. A process for the preparation of a controlled release formulation of erythromycin A or a derivative thereof suitable for once daily administration comprising mixing a pharmaceutically effective amount of erythromycin or a derivative thereof with about 0.1% to about 4% w/w of one or more pharmaceutically acceptable rate controlling polymers.
